(19) United States
(12) Patent Application Publication
(10) Pub. No.: US 2006/0027455 A1
(43) Pub. Date: Feb. 9, 2006

(54) Selective detection of proteins that contain two or more alpha-helical transmembrane domains

(76) Inventors: Wayne F. Patton, Newton, MA (US); Courtenay R. Hart, Eugene, OR (US)

Correspondence Address: KOREN ANDERSON;MOLECULAR PROBES, INC., 29851 WILLOW CREEK ROAD, EUGENE, OR, 97402-9132, US

(21) Appl. No.: 11/199,641

(22) Filed: Aug. 8, 2005

Related U.S. Application Data
(60) Provisional application No. 60/599,339, filed on Aug. 6, 2004.

(51) Int. Cl. C07K 1/26

(52) U.S. Cl. 204/450; 436/86

(57) ABSTRACT

Embodiments of the present invention provide a staining solution and of method of using the staining solution for selectively detecting proteins that contain two or more α-helical transmembrane domains. The staining solution comprises a lipophilic dyes and at least about a 30% hydrophobic solvent. The dyes of the present are represented by the general formula A-B-E wherein A is a nitrogen heterocycle, B is a bridge moiety and E is an electron pair accepting moiety that comprises either a carbonyl or nitrogen atom. In one embodiment these lipophilic dyes are merocyanine dye, a cyanine dye, a styryl dye or a carbazolylvinyl dye.

### CROSS-REFERENCE TO RELATED APPLICATIONS

This application claims priority to U.S. Provisional Patent Application Ser. No. 60/599,339, filed Aug. 6, 2004, which disclosure is herein incorporated by reference.

### STATEMENT REGARDING FEDERALLY SPONSORED RESEARCH

This invention was made in part with government support under grant number R33 CA093292-01, awarded by the National Cancer Institute. The United States Government may have certain rights in this invention.

### FIELD OF THE INVENTION

Lipophilic fluorescent dyes, and to their use in selective detection of proteins that contain two or more α-helical transmembrane domains are disclosed. The dyes and their use have applications in the fields of cell biology, neurology, nutrition, immunology, proteomics and cancer biology.

### BACKGROUND OF THE INVENTION

Integral membrane proteins typically contain one or more hydrophobic, transmembrane domains that intermingle with the hydrophobic portion of lipid bilayer membranes. Given the prominent role that many integral membrane proteins play in signal transduction, they are considered important drug targets for the pharmaceutical industry. However, proteomic profiles generated from two-dimensional gel electrophoresis (2DGE) are well known to lack highly hydrophobic proteins, particularly integral membrane proteins containing more than one α-helical transmembrane domain (Ito, K. and Akiyama, Y. (1985) Biochem. Biophys. Res. Commun. 133: 214-221; Hartinger, J., et al. (1996) Anal. Biochem. 240: 126-133; Ito, K., et al. (1999) Mol Microbiol. 5:1600-1). This is thought to be due to the very poor resolution of this class of proteins in the isoelectric focusing component of the procedure, arising from poor solubilization by nonionic detergents, even in the presence of high concentrations of urea. Should solubilization be achieved initially, the proteins then tend to subsequently precipitate near their isoelectric point. Additionally, this class of proteins is known to display significant isoelectric point heterogeneity due to glycosylation, which results in streaking in the isoelectric focusing dimension.

Conventional SDS-polyacrylamide gel electrophoresis is in many ways more suitable for fractionating integral membrane proteins than 2DGE, due to the excellent solubilization properties of the anionic detergent. However, selective methods for visualization of integral membrane proteins against a background of high abundance hydrophilic cytoplasmic proteins, also present in most biological specimens, have not been devised until now. While general gel staining procedures for the selective detection of hydrophobic proteins have previously been devised using dyes such as Nile Red, Sudan Black B and 8-anilino-1-naphthalene sulfonate (ANS), the methods do not selectively highlight proteins containing multiple transmembrane domains, and are typically used for visualizing serum lipoproteins (Allen, R. C. and Budowle, B. (1999) In: Protein staining and identification techniques. The BioTechniques series on Molecular Laboratory Methods, 3 (BioTechniques Books, Division of Eaton Publishing) pp 82-83.).

Herein we report a novel method for selectively staining integral membrane proteins that combines lipophilic dye compounds and a hydrophobic solvent. The methods readily detect proteins containing two or more transmembrane domains but do not detect serum lipoproteins. After visualizing transmembrane proteins using a laser-based gel scanner, the proteins may be further analyzed such as by staining of the total protein profile with SYPRO® Ruby protein gel stain. The present methods demonstrate a staining intensity that is linear over more than two orders of magnitude of protein concentration. The present methods utilize lipophilic fluorescent dye compounds, such as cyanine, merocyanine and carbazolylvinyl dyes.

The preparation and characterization of cyanine, carbazolylvinyl and merocyanine dyes has been well documented. A large number of useful styryl merocyanine dyes (commonly referred to as RH dyes) have been previously prepared by Rina Hildesheim (Grinvald et al., BIOPHYS. J. 39, 301 (1982)), Leslie Loew (Loew et al., J. ORG. CHEM. 49, 2546 (1984)) and others, as useful probes for measuring electric potentials in cell membranes. Useful membrane potential measurements only occur in live cells and artificial liposomes, where the fluorescence intensity of a suitable dye as it is associated with the membrane changes as the membrane is subjected to an electrical gradient. In addition to the above membrane potential probes, an extensive variety of other merocyanine dyes have been described by Brooker et al. (J. AM. CHEM. SOC. 73, 5326 (1951)), primarily for use in the photographic industry, although Brooker et al. do not describe the fluorescence properties of the merocyanines. Many of these dyes have also been described for detecting proteins when present in an aqueous environment (U.S. Pat. Nos. 5,616,502 and 6,579,718). However, these lipophilic dyes have never been disclosed to be used to selectively detect proteins that contain two or more α-helical transmembrane domains.

### SUMMARY OF THE INVENTION

Embodiments of the present invention provide staining solutions and method of using staining solutions for selectively detecting proteins that contain two or more α-helical transmembrane domains. The staining solutions of the present invention comprise at least one lipophilic dye and at least about 30% (v/v) hydrophobic solvent. The dyes of the present are represented by the general formula A-B-E wherein A is a nitrogen heterocycle, B is a bridge moiety and E is an electron pair accepting moiety that comprises either a carbonyl or nitrogen atom. In one embodiment these lipophilic dyes are merocyanine dye, a cyanine dye, a styryl dye or a carbazolylvinyl dye. Selected dye embodiments include dyes that are represented by Formula VI, VII and IX. A particular advantageous dye for detection of proteins that contain two or more α-helical transmembrane domains when combined with a hydrophobic solvent is Compound 4.

The staining solution for selectively detecting proteins with two or more α-helical transmembrane domains comprises from about 30% (v/v) to about 80% (v/v) hydrophobic solvent and about 0.5 μM to about 5 μM dye. Preferably, the solvent is present between about 40% (v/v) and about 80% (v/v), more preferably the solvent is present at a concentration of from about 45% (v/v) to about 60% (v/v). In one aspect, the solvent is present in the staining solution at about 50% (v/v). Examples of hydrophobic solvents include an alcohol, acetone, acetonitrile, and N-methyl pyrrolidone. Examples of alcohols include methanol, isopropanol, and ethanol.

In one embodiment, the staining solution further comprises an acid at about 5% to about 30%, preferably about 12% to about 20%. In one aspect, the acid is acetic acid. In a particular aspect, the staining solution comprises about 50% (v/v) methanol, about 15% acetic acid and about 2 μM dye.

In an exemplary embodiment, methods for the selective detection of proteins that comprise two or more alpha-helical transmembrane domains in a sample can comprise:
a) contacting a sample with a staining solution to prepare a labeled sample mixture;
b) incubating the labeled sample mixture for a sufficient amount of time to allow a lipophilic dye of the staining solution to non-covalently bind with any transmembranous domains of the protein;
c) illuminating the incubated sample mixture with an appropriate wavelength;
d) observing the illuminated sample mixture whereby one or more proteins that comprise two or more alpha-helical transmembrane domains is detected.

In one aspect, the sample is immobilized on a polymeric membrane, within a polyacrylamide gel, within an agarose gel, on a solid support such as a polymeric membrane or a microarray, before the incubated sample mixture is illuminated.

In another exemplary embodiment, the methods of the present invention for selective detection of proteins that comprise two or more alpha-helical transmembrane domains in a sample, comprise:
a) electrophoretically separating a sample on an SDS-polyacrylamide gel to prepare an immobilized sample;
b) removing the SDS from the polyacrylamide gel to prepare an essentially SDS-free polyacrylamide gel;
c) contacting the essentially SDS-free polyacrylamide gel with a present staining solution to prepare a stained sample;
d) illuminating the stained sample with an appropriate wavelength; and
e) observing the illuminated sample whereby one or more proteins that comprise two or more alpha-helical transmembrane domains is detected.

The sample is prepared and run on the SDS-polyacrylamide gel using standard techniques. Removing the SDS can comprise contacting the sample with a solution such as a fixing solution, wherein the fixing solution comprises an alcohol and an acid. After the gel has been stained, the method can further comprise washing the stained sample to remove unbound dye before the sample has been illuminated.

In another exemplary embodiment, the present invention provides kits for the selective detection of proteins that comprise two or more alpha-helical transmembrane domains in a sample, wherein the kit comprises a present staining solution. In one aspect, the kit further comprises instructions for selective detection of proteins that comprise two or more α-helical transmembrane domains in a sample.

### BRIEF DESCRIPTION OF THE DRAWINGS

FIG. 1: Shows a graph of fluorescence intensity from scanning a gel in which F₁ F₀ ATPase complex was stained with Compound 4 (1A) and then post-stained with SYPRO Ruby total protein gel stain (1B). Subunits α, β, γ, a, δ, b, ε, c are located at 9.8 mm, 11.5 mm, 20.1 mm, 24.3 mm, 26.2 mm, 28.6 mm, 35.1 mm, and 40.2 mm, respectively.

FIG. 2: Shows ratios of Compound 4 signal to SYPRO Ruby total protein gel stain signal for various transmembrane and nontransmembrane proteins. Proteins were separated by SDS-PAGE, stained with Compound 4, imaged, stained with SYPRO Ruby total protein stain and imaged again. The intensity of each band was measured and the ratio of the two signals was plotted as a bar graph. Protein A, bacteriorhodopsin; B, F₀ATPase a subunit; C, F₀ATPase c subunit; D, F₀ATPase b subunit; E, glycophorin; F, porin; G, zein; H, carbonic anhydrase; I, F₁ ATPase α subunit; J, F₁ ATPase β subunit; K, F₁ ATPase γ subunit; L, F₁ ATPase δ subunit; M, F₁ ATPase ε subunit; N, myosin; O, β-galactosidase; P, phosphorylase b; Q, BSA; R, ovalbumin; S, soybean trypsin inhibitor; T, lysozyme; and U, aprotinin. The numbers along the x-axis indicate the number of α helical transmembrane domains present in the corresponding protein; the asterisk denotes a 16-strand anti-parallel β-sheet transmembrane domain.

### DETAILED DESCRIPTION OF THE INVENTION

Introduction

Lipophilic dyes, including styryl dyes, carbazolylvinyl dyes, cyanine dyes, merocyanine dyes, and their derivatives, and their use as selective stains for detecting proteins that comprise two or more α-helical transmembrane domains are described herein.

Aspects of the present invention are unique in that many of the preferred dyes are known as total protein stains in a substantially aqueous environment, but herein are described for their selective staining ability when present in a non-aqueous solvent. Several noteworthy points include:
(1.) Selective detection of proteins comprising 2 or more α-helical transmembrane domains.

The present method does not detect proteins that comprise a single alpha-helical domain, beta-sheet domains, or lipoproteins. Thus, the present method distinguishes between proteins comprising 2 or more α-helical transmembrane domains and other proteins, including other hydrophobic proteins (such as those containing beta-sheets and one α-helical transmembrane domain) and non-hydrophobic proteins.
(2.) Selective staining is achieved by reducing the dye affinity for proteins, so that only the most hydrophobic residues of the protein bind the dye. This is accomplished by performing the dye binding step in a solvent containing a relatively high percentage of organic solvent resulting in a non-aqueous staining solution. Typically the staining solution contains at least about 30% (v/v) of a solvent that is other than water.
(3.) Detection of proteins containing 2 or more α-helical transmembrane domains is accomplished in a wide range of mediums including, but not limited to, in gels, on polymeric membranes, on microarrays, in capillary electrophoresis and in HPLC.

Specific embodiments and preferred embodiments of the lipophilic dyes and methods for detecting proteins comprising 2 or more α-helical transmembrane domains are further described in the detailed description of the invention.

Definitions

Before describing embodiments of the present invention in detail, it is to be understood that this invention is not limited to specific compositions or process steps, and as such may vary. It must be noted that, as used in this specification and the appended claims, the singular form “a”, “an” and “the” includes plural referents unless the context clearly dictates otherwise. Thus, for example, reference to “a fluorescent dye” includes a plurality of dyes and reference to “a compound” includes a plurality of compounds and the like.

Unless defined otherwise, all technical and scientific terms used herein have the same meaning as commonly understood by one of ordinary skill in the art to which this invention is related. The following terms are defined for purposes of the invention as described herein.

Certain compounds can exist in unsolvated forms as well as solvated forms, including hydrated forms. In general, the solvated forms are equivalent to unsolvated forms and are encompassed within the scope of the present invention. Certain compounds may exist in multiple crystalline or amorphous forms. In general, all physical forms are equivalent for the uses contemplated by the present invention and are intended to be within the scope of the present invention.

Certain compounds of the present invention possess asymmetric carbon atoms (sometimes referred to as “optical centers” or “chiral centers”) or double bonds; the racemates, diastereomers, geometric isomers and individual isomers are encompassed within the scope of the present invention.

The compounds disclosed herein may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23:128 (1990).

Although typically not shown for the sake of clarity, any overall positive or negative charges possessed by any of the compounds disclosed herein are balanced by a necessary counterion or counterions. Where the compound is positively charged, the counterion is typically selected from, but not limited to, chloride, bromide, iodide, sulfate, alkanesulfonate, arylsulfonate, phosphate, perchlorate, tetrafluoroborate, tetraarylborate, nitrate, hexafluorophosphate, and anions of aromatic or aliphatic carboxylic acids. Where the compound is negatively charged, the counterion is typically selected from, but not limited to, alkali metal ions, alkaline earth metal ions, transition metal ions, ammonium or substituted ammonium ions. Preferably, any necessary counterion is biologically compatible, is not toxic as used, and does not have a substantially deleterious effect on biomolecules. Counterions are readily changed by methods well known in the art, such as ion-exchange chromatography, or selective precipitation.

The compounds may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty-five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P and S, and wherein the nitrogen, phosphorous and sulfur atoms are optionally oxidized, and the nitrogen heteroatom is optionally be quaternized, and the sulfur atoms are optionally trivalent with alkyl or heteroalkyl substituents. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 4 rings), which are fused together or linked covalently. Specific examples of aryl substituents include, but are not limited to, substituted or unsubstituted derivatives of phenyl, biphenyl, o, m-, or p-terphenyl, 1-naphthyl, 2-naphthyl, 1-, 2-, or 9-anthryl, 1-, 2-, 3-, 4-, or 9-phenanthrenyl and 1-, 2- or 4-pyrenyl. Preferred aryl substituents are phenyl, substituted phenyl, naphthyl or substituted naphthyl.

The term “heteroaryl” as used herein refers to an aryl group as defined above in which one or more carbon atoms have been replaced by a non-carbon atom, especially nitrogen, oxygen, or sulfur. For example, but not as a limitation, such groups include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridaziyl, triazinyl, piperidinyl, morpholinyl, thiomorpholinyl, pyrazinyl, piperainyl, pyrimidinyl, naphthyridinyl, benzofuranyl, benzothienyl, indolyl, indolinyl, indolizinyl, indazolyl, quinolizinyl, qunolinyl, isoquinolinyl, cinnolinyl, phthalazinyl, quinazolinyl, quinoxalinyl, pteridinyl, quinuclidinyl, carbazolyl, acridinyl, phenazinyl, phenothizinyl, phenoxazinyl, purinyl, benzimidazolyl and benzthiazolyl and their aromatic ring-fused analogs. Many fluorophores are comprised of heteroaryl groups and include, without limitations, xanthenes, oxazines, benzazolium derivatives (including cyanines and carbocyanines), borapolyazaindacenes, benzofurans, indoles and quinazolones.

Where a ring substituent is a heteroaryl substituent, it is defined as a 5- or 6-membered heteroaromatic ring that is optionally fused to an additional six-membered aromatic ring(s), or is fused to one 5- or 6-membered heteroaromatic ring. The heteroaromatic rings contain at least 1 and as many as 3 heteroatoms that are selected from the group consisting of O, N or S in any combination. The heteroaryl substituent is bound by a single bond, and is optionally substituted as defined below.

Specific examples of heteroaryl moieties include, but are not limited to, substituted or unsubstituted derivatives of 2- or 3-furanyl; 2- or 3-thienyl; N-, 2- or 3-pyrrolyl; 2- or 3-benzofuranyl; 2- or 3-benzothienyl; N-, 2- or 3-indolyl; 2-, 3- or 4-pyridyl; 2-, 3- or 4-quinolyl; 1-, 3-, or 4-isoquinolyl; 2-, 4-, or 5-(1,3-oxazolyl); 2-benzoxazolyl; 2-, 4-, or 5-(1,3-thiazolyl); 2-benzothiazolyl; 3-, 4-, or 5-isoxazolyl; N-, 2-, or 4-imidazolyl; N-, or 2-benzimidazolyl; 1- or 2-naphthofuranyl; 1- or 2-naphthothienyl; N-, 2- or 3-benzindolyl; 2-, 3-, or 4-benzoquinolyl; 1-, 2-, 3-, or 4-acridinyl. Preferred heteroaryl substituents include substituted or unsubstituted 4-pyridyl, 2-thienyl, 2-pyrrolyl, 2-indolyl, 2-oxazolyl, 2-benzothiazolyl or 2-benzoxazolyl.

The above heterocyclic groups may further include one or more substituents at one or more carbon and/or non-carbon atoms of the heteroaryl group, e.g., alkyl; aryl; heterocycle; halogen; nitro; cyano; hydroxyl, alkoxyl or aryloxyl; thio or mercapto, alkyl- or arylthio; amino, alkyl-, aryl-, dialkyl-, diaryl-, or arylalkylamino; aminocarbonyl, alkylaminocarbonyl, arylaminocarbonyl, dialkylaminocarbonyl, diarylaminocarbonyl or arylalkylaminocarbonyl; carboxyl, or alkyl- or aryloxycarbonyl; aldehyde; aryl- or alkylcarbonyl; iminyl, or aryl- or alkyliminyl; sulfo; alkyl- or arylsulfonyl; hydroximinyl, or aryl- or alkoximinyl. In addition, two or more alkyl substituents may be combined to form fused heterocycle-alkyl ring systems. Substituents including heterocyclic groups (e.g., heteroaryloxy, and heteroaralkylthio) are defined by analogy to the above-described terms.

The term “heterocycloalkyl” as used herein refers to a heterocycle group that is joined to a parent structure by one or more alkyl groups as described above, e.g., 2-piperidylmethyl, and the like. The term “heterocycloalkyl” refers to a heteroaryl group that is joined to a parent structure by one or more alkyl groups as described above, e.g., 2-thienylmethyl, and the like.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R′, —SR′, —halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

The aryl and heteroaryl substituents described herein are unsubstituted or optionally and independently substituted by H, halogen, cyano, sulfonic acid, carboxylic acid, nitro, alkyl, perfluoroalkyl, alkoxy, alkylthio, amino, monoalkylamino, dialkylamino or alkylamido.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “amino” or “amine group” refers to the group —NR′R″ (or NRR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —NRR′R″ and its biologically compatible anionic counterions.

The term “buffer” as used herein refers to a system that acts to minimize the change in acidity or basicity of the solution against addition or depletion of chemical substances.

The term “carbonyl” as used herein refers to the functional group —(C═O)—. However, it will be appreciated that this group may be replaced with other well-known groups that have similar electronic and/or steric character, such as thiocarbonyl (—(C═S)—); sulfinyl (—S(O)—); sulfonyl (—SO₂)—), phosphonyl (—PO₂—).

The term “detectable response” as used herein refers to a change in or an occurrence of, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters. Alternatively, the detectable response is an occurrence of a signal wherein the fluorophore is inherently fluorescent and does not produce a change in signal upon binding to a metal ion. Alternatively, the detectable response is the result of a signal, such as color, fluorescence, radioactivity or another physical property of the detectable label becoming spatially localized in a subset of a sample such as in a gel, on a blot, or an array, in a well of a micoplate, in a microfluidic chamber, or on a microparticle as the result of formation of a ternary complex of the invention that comprises a zinc binding protein.

The term “directly detectable” as used herein refers to the presence of a detectable label or the signal generated from a detectable label that is immediately detectable by observation, instrumentation, or film without requiring chemical modifications or additional substances. For example, a fluorophore produces a directly detectable response.

The term “hydrophobic solvent” as used herein refers to a non-aqueous solvent including solvents that are not miscible in water under ambient conditions of pressure and temperature. This includes, but is not limited to, hydrocarbons (a long list, exemplified by hexane, decane, benzene, toluene, xylene), esters (e.g. ethyl acetate, butyl acetate), ethers (e.g. diethyl ether, dipropyl ether, dibutyl ether), high molecular weight alcohols (starting with n-butanol and larger alcohols, e.g. octanol), heterocycles (e.g. pyridine, quinoline), halogenated solvents (e.g. dichloromethane, chloroform, carbon tetrachloride), and carbon disulfide.

The term “kit” as used herein refers to a packaged set of related components, typically one or more compounds or compositions.

The term “salt thereof,” as used herein includes salts of the agents of the invention and their conjugates, which are preferably prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium, or a similar salt. When compounds of the present invention contain relatively basic functionalities, acid addition salts can be obtained by contacting the neutral form of such compounds with a sufficient amount of the desired acid, either neat or in a suitable inert solvent. Examples of addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “sample” as used herein refers to any material that may contain proteins that contain two or more α-helical transmembrane domains “transmembrane proteins”. Typically, the sample comprises purified or semi-purified transmembrane proteins and endogenous host cell proteins. The transmembrane proteins can be made synthetically or obtained in a purified or semi-purified form from cells (eukaryotic and prokaryotic, without limitation) cell extracts, cell homogenates, or subcellular components as natural or recombinant molecules. Alternatively, the transmembrane proteins can be obtained from tissue homogenate, bodily and other biological fluids, or synthesized proteins, all of which comprise a sample in the present invention. The sample may be in an aqueous or mostly aqueous solution, a viable cell culture or immobilized on a solid or semi solid surface such as a polymer gel, a membrane, a microparticle, an optical fiber or on a microarray.

As used herein the term “sulfonic acid” means either —SO₃H, or a salt of sulfonic acid. Also as used herein the term “carboxylic acid” means either —COOH, or a salt of carboxylic acid. Appropriate salts of sulfonic and carboxylic acids include, among others, K⁺, Na⁺, Cs⁺, Li⁺, Ca²⁺, Mg²⁺, ammonium, alkylammonium or hydroxyalkylammonium salts, or pyridinium salts. Alternatively, the counterion of the sulfonic acid or carboxylic acid may form an inner salt with a positively charged atom on the dye itself, typically a quaternary nitrogen atom.

The Compounds

In general, for ease of understanding the present invention, the lipophilic dye compounds and corresponding substituents will first be described in detail, followed by a staining solution and the method of detecting proteins that comprise two or more α-helical transmenbrane domains, which is followed by exemplified methods of use.

The present methods selectively stain and subsequently detect proteins that comprise two or more α-helical transmembrane domains by combining lipophilic dye compounds in a hydrophobic solvent to form a staining solution. A wide range of lipophilic dyes are envisioned, and there is no intended limitation of the lipophilic dye that can be used with the present methods.

Lipophilic dyes include, but are not limited to, cyanine dyes, merocyanine dyes, styryl dyes and carbazolylvinyl dyes. As used herein “lipophilic” means a dye that comprises a carbon chain that contains at least four, preferably at least five, more preferably at least six, and most preferably at least seven carbons. These carbon chains are present in the form of an alkyl chain and allow the dye to interact with hydrophobic moieties such as lipids, certain detergents such as sodium dodecyl sulfate (SDS) and hydrophobic domains of proteins such as α-helical transmembrane domains. Thus in one aspect of the invention, the methods include preferentially detecting membrane proteins, such as membrane proteins that have 2 or more α-helical transmenbrane domains, by staining proteins with dyes that are known to interact with lipids and detergents such as Sudan black B, Nile Red, 2-(methylanilino)naphthalene-6sulfonic acid and 8-anilino-1-naphthalene sulfonated (ANS) (Sackeft and Wolff (1987) Anal. Biochem 187:228-234; Greenspan and Gutman (1993) Electrophoresis 14:65-68; Silva et al. (1991) Biochem. Internatl. 23:905-913; Sirangrlo et al. (1998) Biochim Biophys Acta 1385:69-77).

While these hydrophobic dyes have been used, with limited success, for the detection of hydrophobic proteins, they have not previously been disclosed for the use of detecting proteins containing two or more α-helical transmembrane domains using methods in which the dyes are dissolved in a staining solution than contains at least about 30% (v/v) of a hydrophobic solvent. The present staining method distinguishes between very hydrophobic proteins (comprising 2 or more α-helical transmembrane domains) and those that are single transmembrane domain proteins and those that contain β-sheets. The present staining method selectively detects those proteins that comprise two or more α-helical transmembrane domains.

By “selectively detects” is meant that, using the disclosed staining methods, the intensity of staining of a protein that contains two or more α-helical transmembrane domains with a hydrophobic dye of the present invention in a hydrophobic solvent, is at least 50%, at least 60%, at least 70%, at least 80% and preferably at least 90% of the intensity of its staining with a dye that stains all proteins. Further, under the staining conditions disclosed for a hydrophobic dye of the present invention in a hydrophobic solvent, the staining intensity ratio (the ratio of staining intensity with a dye of the present invention using the methods disclosed herein and the staining intensity of a dye that binds all proteins for a given protein) of a protein that comprises two or more α-helical transmembrane domains is at least about five-fold, and preferably at least about 10-fold the staining intensity of a protein that does not comprise two or more α-helical transmembrane domains.

While embodiments of the invention are not limited to any particular mechanism, in some aspects of the invention, prior to staining, proteins of a sample can bind SDS, and, after removal SDS from the medium the proteins are in (such as, but not limited to, a gel) hydrophobic proteins having 2 or more α-helical transmembrane domains can continue to bind SDS, while SDS is essentially completely removed from other proteins. For example, SDS can be intercalated into the α-helices of multiple α-helical transmembrane domain proteins such that it is not removed from multiple α-helical transmembrane domain proteins under conditions in which bound SDS is removed from other proteins. In this way, after removing SDS from a sample (such as a sample in a gel) so that a sample is “essentially free of SDS”, a protein having 2 or more α-helical transmembrane domains can be bound by a dye that binds SDS, while other proteins are not bound by the dye. Thus, in addition to the cyanine dyes, merocyanine dyes, styryl dyes and carbazolylvinyl dyes disclosed herein, a dye used in the methods of the present invention that selectively detects those proteins that comprise two or more α-helical transmembrane domains can be any dye that binds a detergent, and, more particularly, any dye that binds SDS.

Many cyanine dyes, merocyanine dyes, styryl dyes and carbazolylvinyl dyes are known and can be used to detect proteins. These dyes, when combined in an aqueous staining solution, can be used as a total protein stain. The term “aqueous solution” as used herein refers to a solution that is predominantly water and retains the solution characteristics of water. Where the aqueous solution contains solvents in addition to water, water is typically the predominant solvent. The present staining solution is not aqueous but rather is hydrophobic because it includes a hydrophobic solvent at greater than or equal to 30% (v/v) of its concentration.

Certain cyanine dyes, merocyanine dyes, styryl dyes and carbazolylvinyl dyes have been commercialized as total proteins stains and sold under the trade names SYPRO Red, SYPRO Orange and SYPRO Tangerine (Molecular Probes, Inc.; Eugene, Oreg.). Unexpectedly, we have shown that these dyes, when combined in a hydrophobic solvent, selectively detect transmembrane proteins, and that prior to or after staining with one of these dyes in a hydrophobic solvent, the total protein profile in the sample can be stained using a total protein stains. This allows for multiplexing, but more importantly demonstrates that the same dye, in a different environment, aqueous as compared to hydrophobic, can be used to detect a select subset of proteins.

Thus, aspects of the present invention includes without limitation, lipophilic dyes such as cyanine dyes, merocyanine dyes, styryl dyes and carbazolylvinyl dyes and any dye disclosed in U.S. Pat. Nos. 6,579,718; 5,616,502; 5,436,134; 5,656,449; 5,658,751; 6,004,536; 4,883,867 and 4,957,870. It is understood that a larger number of lipophilic dyes have been previously disclosed and that the invention is not limited to those dyes disclosed in the above patent references but includes all known lipophilic cyanine dyes, merocyanine dyes, styryl dyes and carbazolylvinyl dyes, and those invented in the future.

Cyanine, styryl, carbazolylvinyl, and merocyanine dyes are a diverse group of dyes that comprise a quaternary nitrogen heterocycle linked to an electron pair-donating moiety by an alkylene or polyalkylene bridge. Thus, in an exemplary embodiment, the present lipophilic dyes are represented by the general formula A-B-E wherein A is a nitrogen heterocycle, B is a bridge moiety; and E is an electron pair accepting moiety that comprises either a carbonyl or nitrogen atom.

In an exemplary embodiment, A is a quaternized nitrogen heterocycle where the quaternizing group is R² and A is represented by the formulas:

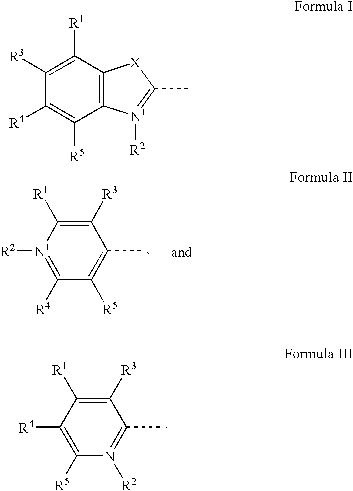

The quarternizing nitrogen substituent R² is alkyl, substituted alkyl, sulfoalkyl, substituted sulfoalkyl, aminoalkyl or substituted aminoalkyl. R² is typically a sulfoalkyl, aminoalkyl or a substituted aminoalkyl wherein the amino group is substituted with an alkyl, aminoalkyl or sulfoalkyl.

In an exemplary embodiment, R² includes at least one nitrogen heteroatom, preferably wherein the nitrogen atom is a dialkylamino or a trialkylammonium substituent, and where the alkyl substituents are methyl or ethyl. In another embodiment, R² is —CH₃, or CH₂CH₃, or R² is a C₃-C_{C}₂₂ alkyl chain that is linear or branched, saturated or unsaturated, and that is optionally substituted one or more times by hydroxy, carboxy, sulfo, amino, amino substituted by 1-2 C₁-C₆ alkyls, or ammonium substituted by 1-3 C₁-C₆ alkyls. In one aspect, R² is a C₃-C₁₂ alkyl chain that is linear and saturated, and substituted at its free terminus by hydroxy, carboxy, sulfo, amino, amino substituted by 1-2 C₁-C₆ alkyls, or ammonium substituted by 1-3 C₁-C₆ alkyls. In another aspect R² is a C₃-C₄ alkyl that is substituted once by sulfo or carboxy.

Alternatively, the nitrogen atoms of R² form either one or two saturated 5- or 6-membered rings in combination with other C or N atoms in R², such that the resulting rings are pyrrolidines, piperidines, piperazines or morpholines.

The aromatic substitutents R¹, R³, R⁴, R⁵ are independently hydrogen, halogen, substituted halogen, alkyl, substituted alkyl, sulfoalkyl, alkoxy, substituted alkoxy, amino, substituted amino, aminoalkyl or substituted aminoalkyl. Alternatively, the aromatic ring can be fused to additional rings wherein a member independently selected from; R¹ in combination with R³; R³ in combination with R⁴; and R⁴ in combination with R⁵; together with the atoms to which they are joined, form a ring which is a 5-, 6- or 7-membered cycloalkyl, a 5-, 6- or 7-membered heterocycloalkyl, a 5-, 6- or 7-membered aryl or a 5-, 6- or 7-membered heteroaryl (yielding a benzo-substituted pyridinium, or quinolinium moiety).

The additional ring on the quinolinium that is thereby formed is optionally and independently substituted one or more times by halogen, alkyl, perfluoroalkyl, alkoxy, amino, or amino substituted by alkyls. Additionally, the quinolinium ring is optionally substituted by an additional fused 6-membered aromatic ring (yielding a naphtho-substituted pyridinium, or a benzoquinoline), that is also optionally and independently substituted one or more times by halogen, alkyl, perfluoroalkyl, alkoxy, amino, or amino substituted by alkyls. Typically, R¹ and R³ are hydrogen, or form a substituted or unsubstituted benzo moiety.

In the benzazole ring (Formula I), the ring fragment X is O, S, NR⁵, or CR¹¹R¹² wherein R⁵ is disclosed above and R¹¹ and R¹² are independently hydrogen, halogen, phenyl, substituted phenyl, substituted halogen, alkyl, or substituted alkyl or R¹¹ and R¹² in combination form a 5- or 6-membered ring. When X is CR¹R ¹², R¹¹ and R¹² are typically hydrogen. Typically X is O or S, more typically X is O.

B is a covalent bridge that is an alkylene or polyalkylene covalent linkage that is generally referred to as a methine bridge. B has the formula —(CR¹¹═CR¹²)ₙ- wherein R¹¹ and R¹² are independently hydrogen, halogen, phenyl, substituted phenyl, substituted halogen, alkyl, or substituted alkyl. In one aspect, R¹¹ and R¹² are hydrogen.

The value n is a positive, non-zero integer, and determines how many conjugated alkenyl moieties are joined to form the bridge. For example, n can be 1, 2, or 3. The spectral properties of the resulting dye are highly dependent upon the length of the bridge moiety, with the excitation and emission wavelengths shifting to longer wavelengths with the addition of each alkenyl moiety. Thus, when selecting dyes, compounds with longer methine bridges, wherein n is 2 or 3 will typically have a longer emission wavelength than those compounds wherein n is 1.

A wide variety of electron pair-donating groups are known that stabilize the formally positive charge of the quaternary nitrogen heterocycle by resonance. Suitable electron pair-donating groups include dialkylaminophenyl, dialkylaminonaphthyl, electron-rich heterocycles and acyclic moieties containing electron pair-donating groups.

In an exemplary embodiment, E is an aromatic heterocyclic substituent or activated methylene substituent. In a further embodiment E is represented by the formula:

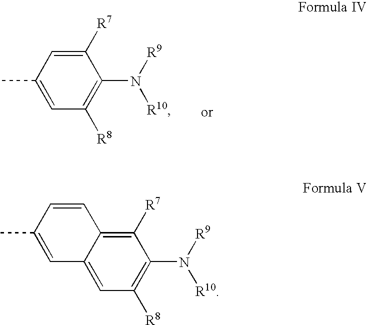

The aromatic substituents R⁷ and R⁸ of Formula IV and V are independently hydrogen, halogen, substituted halogen, alkyl, substituted alkyl, sulfoalkyl, amino, substituted amino, aminoalkyl or substituted aminoalkyl. In an exemplary embodiment, R⁷ and R⁸ are hydrogen.

The amino substituents R⁹ and R¹⁰ are independently alkyl, substituted alkyl, sulfoalkyl, aminoalkyl or substituted aminoalkyl. In one embodiment R⁹ and R¹⁰ are C₁-C₁₈ alkyls that are linear, branched, saturated or unsaturated, and are optionally substituted one or more times by halogen, hydroxy or alkoxy. In a further aspect, R⁹ and R¹⁰ are each linear C₄-C₈ alkyls, preferably R⁹ and R¹⁰ are C₅-C₇ alkyls. Alternatively, R⁹ and R¹⁰ in combination form a 5- or 6-membered ring; R⁹ and R⁷ in combination for a 5- or 6-membered ring or R¹⁰ and R⁸ in combination form a 5- or 6-membered ring. In one embodiment the formed ring contains an oxygen heteroatom.

In a particularly preferred embodiment, at least one of R⁹ and R¹⁰ (or both) contain a lipophilic alkyl moiety wherein the alkyl portion contains at least four carbons.

In an exemplary embodiment, the lipophilic dyes are represented by the general formula

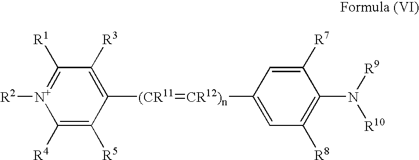

Wherein R¹, R², R³, R⁴, R⁵, R⁷, R⁸, R⁹, R¹⁰, R¹¹ and R¹² are defined above.

A particularly preferred dye of the present invention is Compound 4 that is represented by the structure:

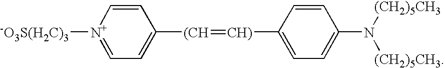

In a further embodiment, wherein R⁴ and R⁵ form a 6-membered fused ring, the lipophilic dyes are represented by the formula:

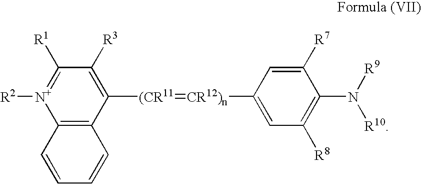

Wherein R¹, R², R³, R⁷, R⁸, R⁹, R¹⁰, R¹¹ and R¹² are defined above.

A particularly preferred dye of the present invention is Compound 5 that is represented by the structure:

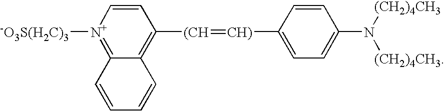

In another exemplary embodiment, E is a substituted or unsubstituted carbazolyl moiety attached at its 3-position to B that is an ethenyl (vinyl) or polyethenyl/alkylene or polyalkylene bridging moiety. In one aspect, E is represented by the formula:

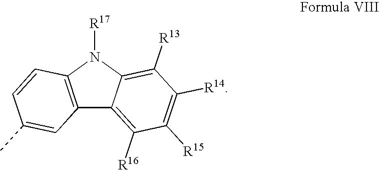

The aromatic substituents R¹³, R¹⁴, R¹⁵, and R¹⁶ are independently hydrogen, halogen, substituted halogen, alkyl, substituted alkyl, sulfoalkyl, alkoxy, substituted alkoxy, amino, substituted amino, aminoalkyl or substituted aminoalkyl.

The nitrogen substituent R¹⁷ of Formula VI is alkyl, substituted alkyl, phenyl, substituted phenyl, amino alkyl, or substituted aminoalkyl. In one embodiment R¹⁷ is methyl, ethyl or phenyl. In another embodiment R¹⁷ is a sulfoalkyl or an aminoalkyl wherein the amino group is optionally substituted by an alkyl group or an aminoalkyl group.

In an exemplary embodiment, the lipophilic dyes are represented by the general formula:

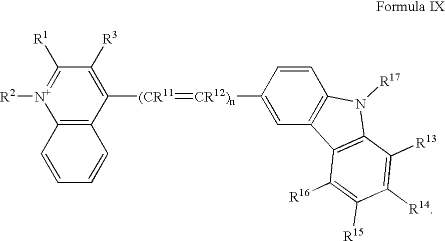

Wherein R¹, R² R³, R¹¹, R¹², R¹³, R¹⁴, R¹⁵, R¹⁶ and R¹⁷ are defined above.

A particularly preferred dye of the present invention is Compound 6 that is represented by the structure:

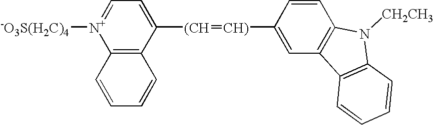

Syntheses of many of the preferred embodiments of the dyes are well documented including in the following references (U.S. Pat. Nos. 5,616,502 and 6,579,718; Grinvald et al., BIOPHYS. J. 39, 301 (1982); Loew et al., J. ORG. CHEM. 49, 2546 (1984); and Brooker et al. (J. AM. CHEM. SOC. 73, 5326 (1951)).

Staining Solution

The present staining solution for selectively detecting proteins that comprise two or more alpha-helical transmembrane domains in a sample comprises:

a. a lipophilic dye compound; and

b. at least about 30% (v/v) hydrophobic solvent.

The lipophilic dyes described above, including SDS-binding dyes, specifically or generically, can be used in the present staining solution. For fluorescence detection, dye concentrations are typically greater than 0.10 μM and less than 10 μM; preferably greater than about 0.50 μM and less than or equal to about 5 μM; more preferably about 1 μM to about 3 μM. In one aspect, the concentration of the dye in the staining solution is about 2 μM. Although concentrations below and above these values likewise result in detectable staining for transmembrane proteins, depending on the sensitivity of the detection method, dye concentrations greater than about 10 μM generally lead to some quenching of the fluorescence signal.

A particular dye is generally selected for a particular assay using one or more of the following criteria: domain selectivity, sensitivity to poly(amino acids), insensitivity to the presence of nucleic acids, dynamic range, photostability, staining time, and spectral properties. The sensitivity and dynamic range of the dyes is determined using the procedures of Example 3. Preferably, the dyes have a sensitivity of 10 ng or less of transmembrane proteins per band in electrophoretic gels. The preferred dyes have a dynamic range of about 2 or more orders of magnitude of transmembrane protein concentration for immobilized assays.

To make a staining solution to combine with the sample, the selected dye is typically first dissolved in an organic solvent, such as DMSO, DMF or methanol, usually to a dye concentration of 1-10 mM. This concentrated stock solution is then generally diluted in a non-aqueous that is typically different from the organic solvent used to make the stock solution. The present staining solution may contain an aqueous component but the staining solution is not considered aqueous due to the small volume of water present and the larger percentage of a non-aqueous or hydrophobic solvent. The non-aqueous solvent is present in the staining solution at least about 30% (v/v), more preferably from about 40% to about 80%, even more preferred the solvent is present from about 45% to about 60%. In an exemplary embodiment, the solvent is present at about 50% (v/v).

Non-aqueous solvents or hydrophobic solvents, include but are not limited to, hydrocarbons (a long list, exemplified by hexane, decane, benzene, toluene, xylene), esters (e.g. ethyl acetate, butyl acetate), ethers (e.g. diethyl ether, dipropyl ether, dibutyl ether), high molecular weight alcohols (starting with n-butanol and larger alcohols, e.g. octanol), heterocycles (e.g. pyridine, quinoline), halogenated solvents (e.g. dichloromethane, chloroform, carbon tetrachloride), carbon disulfide, acetone, ethanol, methanol, isopropnol acetonitrile, and N-methyl pyrrolidone. In an exemplary embodiment, the solvent is acetone, ethanol, methanol, isopropnol acetonitrile, and N-methyl pyrrolidone. Acetone, methanol, and acetonitrile are equally preferred for the dyes that were tested, See Example 3. In a particularly exemplified embodiment the staining solution contains 50% (v/v) methanol.

In further embodiments, the staining solution may contain buffering components such as, 50-100 mM formate buffer, pH 4.0, sodium citrate, pH 4.5, sodium acetate, pH 5.0, MES, pH 6.0, imidazole, pH 7.0, HEPES, pH 6.8, Tris acetate, pH 8.0, Tris-HCl, pH 8.5, Tris borate, pH 9.0 and sodium bicarbonate, pH 10.

In another embodiment, the staining solution contains an acid component. Typical suitable acidic components include without limitation acetic acid, trichloroacetic acid, trifluoroacetic acid, perchloric acid, or sulfuric acid. The acidic component is typically present at a concentration of about 1%-20% and is buffered to the appropriate pH by a base. In one aspect, the acid is acetic acid, which is typically present in a concentration of about 5% to about 20%. In a further aspect, the staining solution contains about 15% acetic acid.

In an exemplary embodiment, the staining solution comprises about 40% (v/v) to about 75% (v/v) non-aqueous or hydrophobic solvent and about 0.5 μM to about 5 μM dye. In one aspect, the staining solution comprises about 10% to about 30% acid. Preferably the acid component is acetic acid. Alternatively, instead of acetic acid the staining solution comprises HEPES at about pH 6.8.

In a further embodiment, the solvent is alcohol, typically ethanol, isopropanol or methanol. In one aspect, the solvent is methanol and is present about 50% (v/v).

In a particularly advantageous embodiment, the staining solution contains about 50% (v/v) methanol, about 15% acetic acid and about 2 μM dye. In one aspect, the dye is Compound 4, Compound 5 or Compound 6. Particularly preferred is Compound 4, especially for detecting proteins comprising two or more α-helical transmembrane domains in a polyacrylamide gel.

Methods of Use

Embodiments of the present invention use the staining solution described above to stain proteins that comprise 2 or more α-helical transmembrane domains, followed by detection of the stained transmembrane proteins and optionally their quantification. Additional steps are optionally and independently used, in any combination, to provide for separation or purification of the transmembrane proteins, for enhancing the detection of the transmembrane proteins, or for quantification of the transmembrane proteins.

In an exemplary embodiment, a method for the selective detection of proteins that comprise two or more alpha-helical transmembrane domains in a sample, comprises the following steps:
a) contacting the sample with a present staining solution to prepare a labeled sample mixture;
b) incubating the labeled sample mixture for a sufficient amount of time to allow a lipophilic dye of the staining solution to non-covalently bind with any transmembraneous domains of the protein;
c) illuminating the incubated sample mixture with an appropriate wavelength;
d) observing the illuminated sample mixture whereby the protein that comprises two or more alpha-helical transmembrane domains is detected.

The staining solution is combined with the sample in such a way as to facilitate contact between the lipophilic dye, and any transmembraneous domains of the proteins present in the sample. When the sample is immobilized on a solid or semi-solid support, the staining solution is typically incubated with the sample under conditions that maximize contact, such as gentle mixing or rocking.

The labeled sample mixture is typically incubated for less than about 12 hours, typically less than about 8 hours, more typically less than about 4 hours. In one aspect, the sample and staining solution are incubated less than about 3 hours and in a further aspect the sample and staining solution are incubated about 2 hours.

In an exemplary embodiment, the sample is immobilized before the staining solution and the sample are incubated. In this way discrete transmembrane proteins can be detected and optionally isolated using standard isolation techniques. Typically the sample is separated on a gel, typically a SDS-polyacrylamide gel. Alternatively, the sample is immobilized on solid or semi-solid matrix that includes a membrane, polymeric beads, polymeric gel, a glass surface or an array surface.

Therefore, in an exemplary embodiment, a method for selective detection of proteins that contain two or more alpha-helical transmembrane domains in a sample, wherein the method comprises:
a) electrophoretically separating the sample on an SDS-polyacrylamide gel to prepare an immobilized sample;
b) removing the SDS from the polyacrylamide gel to prepare an essentially SDS-free polyacrylamide gel;
c) contacting the essentially SDS-free polyacrylamide gel with a present staining solution to prepare a stained sample;
d) illuminating the stained sample with an appropriate wavelength; and
e) observing the illuminated sample whereby the protein that contains two or more alpha-helical transmembrane domains is detected.

In step one (a) a sample, obtained as described below, is prepared in an appropriate buffer and separated on a SDS-polyacrylamide gel using standard SDS-polyacrylamide gel electrophoresis techniques. An appropriate buffer includes a SDS-sample buffer containing Tris, glycerol, DTT, SDS, and bromophenol blue. We have found that for qualitative improvements in staining that an acetone or chloroform-methanol precipitation of the proteins prior to electrophoresis can be performed.

When SDS-polyacrylamide gels are employed, the denaturing effects of the SDS buffer allow for the exposure of hydrophobic regions that might otherwise be obscured from lipophilic dyes that have affinity for hydrophobic domains by protein folding. Thus, SDS gel electrophoresis facilitates the binding of the lipophilic dyes of the present invention with the proteins comprisng two or more α-helical transmebrane domains. However, the lipophilic dyes of the invention also typically bind to SDS. Thus, it is important that after the sample has been separated that the SDS be removed from the gel with a fixing solution for maximal detection of proteins comprising two or more α-helical transmembrane domains. This results in an essentially SDS-free gel that when contacted with a present staining solution allows for detection of target proteins with a fluorescence enhancement of more than 5× compared to proteins that comprise no or one α-helical transmembrane domains. Preferably the fluorescence enhancement is more than about 10×, more preferably more than about 20× and even more preferably more than about 40×.

Therefore, the second (b) step comprises removing the SDS from the polyacrylamide gel to prepare an essentially SDS-free polyacrylamide gel. An essentially SDS-free polyacrylamide gel does not display background staining of the gel or nonhydrophobic membranes separated in the gel with an SDS-binding dye. Without wishing to be bound to a theory it is possible that after removal of SDS from the gel (such as by incubating in a fixing solution), the SDS associated with the α-helical transmembrane domains is not removed from multiple α-helical domain hydrophobic proteins, and as such the lipophilic dyes in the staining solution that bind the multiple α-helical transmembrane domain proteins are binding to the intercalated SDS.

In an alternative scenario, the SDS could be entirely removed from the multiple α-helical transmembrane domain proteins by a fixing solution and the lipophilic dyes may bind with the hydrophobic α-helical transmembrane domains, which are exposed by the denaturing effects of the SDS-gel.

The fixing solution contains a polar organic solvent, typically an alcohol. Preferably, the polar organic solvent is an alcohol having 1-6 carbon atoms, or a diol or triol having 2-6 carbon atoms. Preferred alcohols are methanol or ethanol mixed with acetic acid. The alcohols are present in an aqueous solution of about 50% (v/v) ethanol or methanol with 10% acetic acid. Fixing solutions containing less than 50% (v/v) of ethanol or methanol generally result in incomplete removal of SDS from the gels.

To remove the SDS coat from the gel and immobilized proteins, the polyacrylamide gel is incubated in the fixing solution. Preferably the gel is fixed in multiple sequential steps, typically two. Essentially, the gels is immersed in the fixing solution for at least 20 minutes and then removed from the solution and new solution added for at least 3 hours and up to 24 hours. Generally, one step of incubating the gel in fixing solution is insufficient to remove essentially all of the SDS from the gel.

During the third (c) step the essentially SDS-free polyacrylamide gel is contacted with a staining solution of the present invention. As described above, the gel is typically incubated in the staining gel for sufficient amount of time for the dye to bind protein comprising two or more alpha-helical transmembrane domains, for example, for at least about one hour and less than about 12 hours, preferably less than 8 hours, more preferably less than 4 hours. Most preferred the gel is incubated with the staining solution for at least about one hour and less than about 2.5 hours. Preferably the gel incubating in the staining solution is placed in the dark at room temperature with gentle agitation.

Preferably, the gel is destained briefly, for example, in a destain solution comprising a low percentage of an alcohol and a weak acid. An example of a destain solution is 5% (v/v) methanol, 5% acetic acid. Destaining can be for 1 to 30 minutes, and is preferably for 2 to 10 minutes, for example 5 minutes. Two or more destainings can be performed in succession. The gel is preferably rinsed after destaining with water to remove unbound fluorescent dye prior to illumination. The rinse can be an incubation of from 5 minutes to an hour. Multiple rinses can be performed. In one example, a gel can be destained twice for 5 minutes, and then rinsed in water two to three times for about 30 minutes each. Destaining steps and rinse steps can vary.

The selection of the dye dictates the appropriate wavelength for excitation and subsequently emission resulting in a detectable signal.

The sample is optionally combined with one or more other solutions in the course of staining, including wash solutions, permeabilization and/or fixation solutions, and solutions containing additional detection reagents. An additional detection reagent typically produces a detectable response due to the presence of a specific analyte, such as total proteins or a subset such as phosphoproteins glycoproteins or fusion proteins containing an epitope or affinity tag.

After detection of proteins comprising two or more α-helical transmembrane domains, the gel may be stained for a total protein profile using the commercially available SYPRO Ruby total protein stain (Molecular Probes, Inc.; Eugene, Oreg.) Alternatively, before the SDS has been removed from the gel the commercially available SYPRO Orange, SYPRO Red, SYPRO Tangerine total protein stains (Molecular Probes, Inc.; Eugene, Oreg.) can be used to stain the total protein profile. The gels can then be washed to remove the stain, SDS removed and the gels re-stained using a staining solution of the present invention to detect proteins having two or more α-helical transmembrane domains. Furthermore, where the additional detection reagent has, or yields a product with, spectral properties that differ from those of the subject dye compounds, multi-color applications are possible. This is particularly useful where the additional detection reagent is a dye or dye-conjugate of the present invention having spectral properties that are detectably distinct from those of the staining dye.

Sample Preparation

The sample is any medium suspected to contain a protein that contains two or more α-helical transmembrane domains. The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells such as bacteria, yeast, fungi, mycobacteria and mycoplasma, and eukaryotic cells such as nucleated plant and animal cells that include primary cultures and immortalized cell lines. Typically prokaryotic cells include E. coli and S. aureus. Eukaryotic cells include without limitation ovary cells, epithelial cells, circulating immune cells, β cells, hepatocytes, and neurons.

In yet another embodiment, the sample is present on or in solid or semi-solid matrix. In one aspect, of the invention, the matrix is a membrane. In another aspect, the matrix is an electrophoretic gel, such as is used for separating and characterizing nucleic acids or proteins. In another aspect, the matrix is a silicon chip or glass slide, and the analyte of interest has been immobilized on the chip or slide in an array. In yet another aspect, the matrix is a microwell plate or microfluidic chip, and the sample is analyzed by automated methods, typically by various methods of high-throughput screening, such as drug screening.

Illumination

At any time after or during staining, the sample is illuminated with a wavelength of light selected to give a detectable optical response, and observed with a means for detecting the optical response. Equipment that is useful for illuminating the dye compounds of the invention includes, but is not limited to, ultraviolet lamps, mercury arc lamps, xenon lamps, lasers and laser diodes. These illumination sources are optionally integrated into laser scanners, fluorescence microplate readers, standard or minifluorometers, or chromatographic detectors. The optical response is optionally detected by visual inspection, or by use of any of the following devices: CCD cameras, video cameras, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. A detectable optical response means a change in, or occurrence of, an optical signal that is detectable either by observation or instrumentally. Typically the detectable response is a change in fluorescence, such as a change in the intensity, excitation or emission wavelength distribution of fluorescence, fluorescence lifetime, fluorescence polarization, or a combination thereof.

In particular, present lipophilic dyes can be selected that possess excellent correspondence of their excitation band with the 488 nm band of the commonly used argon laser or emission bands which are coincident with preexisting filters.

Kits

Suitable kits for selectively detecting proteins that comprise two or more α-helical transmembrane domains also form part of the invention. Such kits can be prepared from readily available materials and reagents and can come in a variety of embodiments. The contents of the kit will depend on the design of the assay protocol or reagent for detection or measurement. All kits will contain instructions, appropriate reagents, and staining solution. Typically, instructions include a tangible expression describing the reagent concentration or at least one assay method parameter such as the relative amounts of reagent and sample to be added together, maintenance time periods for reagent/sample admixtures, temperature, buffer conditions and the like to allow the user to carry out any one of the methods or preparations described above. Kits can also include one or more proteins that that comprise two or more α-helical transmembrane domains that can be used as staining or size standards.

Thus, in an exemplary embodiment, a kit comprises a present staining solution. In a further aspect the kits contains additional detection reagents. In this instance additional detection reagents include, but are not limited to, total protein stains, domain selective protein stains, antibodies and other selective detection reagents.

A detailed description of the invention having been provided above, the following examples are given for the purpose of illustrating the invention and shall not be construed as being a limitation on the scope of the invention or claims.

### EXAMPLES

### Example 1

### Selective Staining of Proteins Containing Two or More Alpha Helical Transmembrane Domains in the Protein Complex F₁F₀ ATPase After Separation by SDS Polyacrylamide Gel Electrophoresis

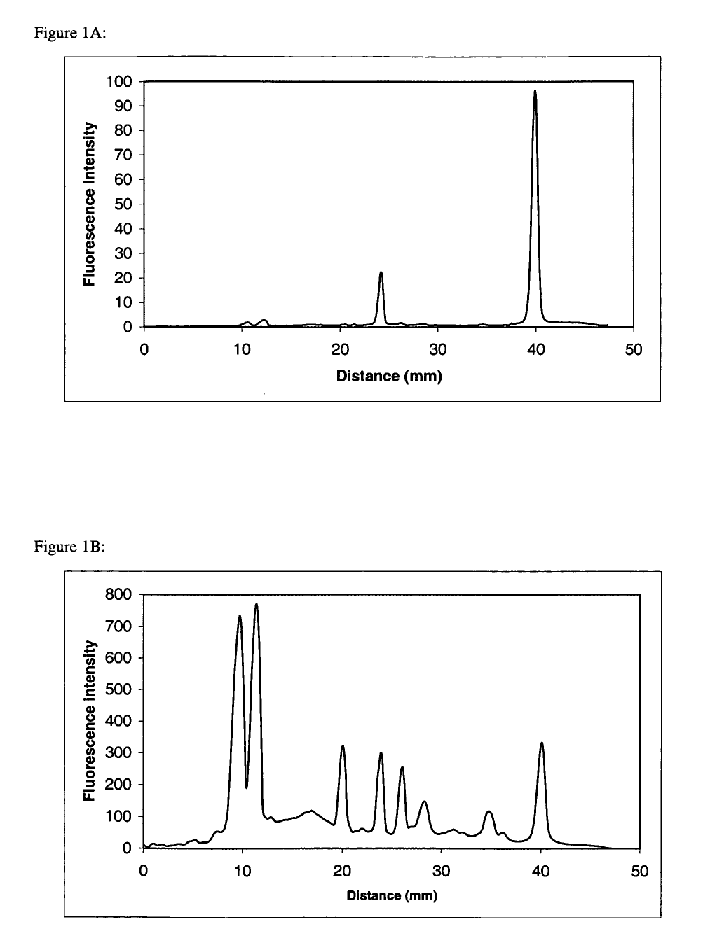

The protein complex F₁F₀ ATPase consists of a water-soluble F₁-ATPase complex of 5 subunits (α, β, γ, δ, ε) and an insoluble membrane-embedded F₀ complex of 3 subunits (a,b, c). F₁ ATPase does not contain proteins having transmembrane domains, while F₀ a, b, and c subunits contain a 5,1, and 2 α-helical transmembrane domains, respectively. Purified F₁F₀ ATPase was dissolved in 1× SDS sample buffer (50 mM Tris, 10% glycerol, 100 mM DTT, 2% SDS, 0.2% bromophenol blue, pH 6.8). Proteins were separated by SDS-polyacrylamide gel electrophoresis utilizing 10-20% acrylamide Tris-glycine precast Ready gels (Bio-Rad Laboratories, Hercules, Calif.). The 1 mm thick, 8×8 cm gels were subjected to electrophoresis using the Bio-Rad Mini Protean III system according to standard procedures. Following separation, the gels were fixed 20 minutes in 100 ml of 50% ethanol/7% acetic acid and then fixed overnight in fresh fixative solution. The gels were next incubated in a staining solution containing 2 μM Compound 4 in 50% methanol, 15% acetic acid for 2 hours in a total volume of 50 ml. Afterwards, the gels were washed two times for 5 minutes each in 50 ml of 5% methanol, 5% acetic acid and then washed 2 times for 30 minutes each in deionized water. Fix and wash volumes were approximately 100 mL. All incubation and wash steps were performed with gentle orbital shaking, typically at 50 rpm. Stained gels were protected from bright light exposure by covering with aluminum foil. The resulting orange-fluorescent signal produced by the Compound 4 was visualized using the 473 nm excitation line of the SHG laser on the Fuji FLA-3000G Fluorescence Image Analyzer (Fuji Photo, Tokyo, Japan) with a 520 nm long pass emission filter. The intensity profile analysis provided in FIG. 1A shows two peaks of high intensity, corresponding to F₀ subunits a and c, the five and two transmembrane domain-containing proteins present in the F₁F₀ complex.

The gel was subsequently incubated overnight in 75 ml of SYPRO Ruby total protein gel stain (Molecular Probes, Inc.). The gel was then washed in 50 ml of 7% acetic acid, 10% methanol for 30 minutes and then with deionized water for 30 minutes. The resulting red fluorescent signal was visualized using the 473 nm excitation line of the SHG laser on the Fuji FLA-3000G Fluorescence Image Analyzer with a 580 nm long pass emission filter. Post-staining of the gel with SYPRO Ruby dye shows eight peaks, corresponding to all the proteins present in the F₁F₀ complex, provided in FIG. 1B.

### Example 2

### Selective Staining of Proteins Containing Two or More Alpha Helical Transmembrane Domains After Separation by SDS Polyacrylamide Gel Electrophoresis

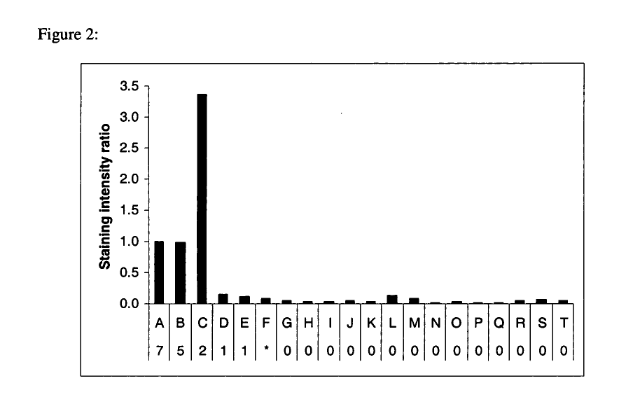

Representative examples of hydrophobic proteins containing no transmembrane domains, α helical transmembrane domains and a β sheet transmembrane domain along with water soluble proteins were prepared in 1×SDS sample buffer, separated by SDS-polyacrylamide gel electrophoresis, stained with Compound 4, imaged, stained for total proteins with SYPRO Ruby dye, and imaged again as described in Example 1. The intensity of the signals from Compound 4 and SYPRO Ruby stain was measured using ImageGauge software (Fuji Photo, Tokyo, Japan) for each protein and the ratio of the two signals was calculated and normalized for bacteriorhodopsin, a seven a helical transmembrane domain protein (FIG. 2). Proteins containing two or more a helical transmembrane domains were preferentially stained.

### Example 3

### Selective Staining of Proteins Containing Two or More Alpha Helical Transmembrane Domains in the Protein Complex F₁F₀ ATPase After Separation by SDS Polyacrylamide Gel Electrophoresis

Bacteriorhodopsin, a seven α-helical transmembrane domain-containing protein, F₁F₀ ATPase complex, and a protein marker mixture containing myosin, β-galactosidase, phosphorylase b, BSA, ovalbumin, carbonic anhydrase, soybean trypsin inhibitor, lysozyme, and aprotinin (all non-transmembrane domain proteins) were prepared in 1× LDS sample buffer (50 mM Tris, 10% glycerol, 50 mM DTT, 2% LDS, 0.015% EDTA, 0.1% Serva Blue G250, and 0.1% Phenol Red, pH 8.5). Proteins were separated by SDS-polyacrylamide gel electrophoresis utilizing 4-12% acrylamide Bis-Tris NuPAGE precast gels (Invitrogen, Carlsbad, Calif.). The 1 mm thick, 8×8 cm gels were subjected to electrophoresis using the NuPAGE XCell Mini-Cell system according to standard procedures. Following separation, the gels were fixed 20 minutes in 100 ml of 50% ethanol/7% acetic acid and then overnight in 100 ml of fresh fixative solution. Gels were next washed two times for 20 minutes each in deionized water. The gels were then incubated in a staining solution containing 2 μM of a candidate dye in 40% acetonitrile, 15% acetic acid for 2 hours in a total volume of 50 ml. Afterwards, the gels were washed two times for 5 minutes each in 50 ml of 5% acetonitrile, 5% acetic acid and then washed 2 hours in approximately 100 mL deionized water. All incubation and wash steps were performed with gentle orbital shaking, typically at 50 rpm. Stained gels were protected from bright light exposure by covering with aluminum foil. The gels were imaged, post-stained for total proteins with SYPRO Ruby total protein stain and imaged again as given in Example 1. Following staining with a candidate dye the proteins bacteriorhodopsin, F₀ a subunit, and F₀ c subunit were analyzed to determine the dyes that selective stained these proteins. Following staining with SYPRO Ruby dye all proteins listed above were prominently visible.

This protocol described herein was used to screen a wide class of compounds to determine those that selectively detected proteins that contained two or more α-helical transmembrane domains. See, Table 1.

TABLE 1
Transmembrane protein dye screen

| | | Transmembrane | |
|---|---|---|---|
| Compound | Optimal | Protein | |
| #² | Ex | Specific?¹ | Relative Intensity |
| Compound 4 | 473 | Yes | Bright |
| Compound 5 | 532 | Yes | Bright |
| Compound 6 | 473 | Yes | Moderate, high background |
| Compound 8 | 473 | Yes | Dim |
| Compound 9 | 488 | Yes | Dim |
| Compound 10 | 488 | Yes | Dim |
| Compound 11 | 488 | Complex, TP | Moderate |
| Compound 12 | 488 | Complex, TP | Moderate-bright |
| Compound 13 | 488 | Complex, TP | Bright |
| Compound 14 | 488 | Complex, TP | Very Bright |
| Compound 15 | 473 | Complex | Bright |
| Compound 16 | 532 | Complex | Dim |
| Compound 17 | 473 | No | Bright |
| Compound 18 | 473 | No | Dim |
| Compound 19 | 488 | Total Protein | Bright |
| Compound 20 | 473 | No | Moderate, high background |
| Compound 21 | 473 | No | Bright |

¹Specificity was determined based on preferential staining of transmebrane
proteins a and c subunits of F0 and bacterorhodapsin. Yes: F0 a and c
subunit staining brighter than F₁ α and β subunits. No: F1 α and
β subunits more strongly stained than F0 a and c subunits. Total
Protein: general total protein stain, even low concentration nonspecific
proteins detected.
Complex: nontransmembrane proteins appear hollow (quenched) in
center of band and transmembrane proteins are positively stained.
²See, Structure of Compounds below.

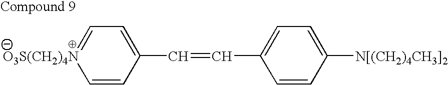

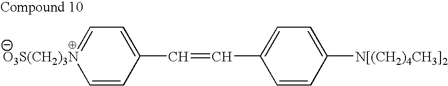

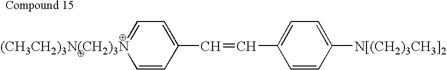

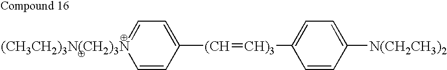

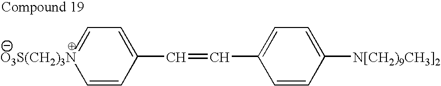

### Example 4

### Synthesis of Compound 20

A mixture of 1.11 g of 9-ethyl-carbazolecarboxaldehyde, 1.1 g of 1-(3-sulfopropyl)-4-methylpyridinium, inner salt and 0.15 mL of piperidine is heated overnight in 25 mL of ethanol at 60 C. Product is collected by filtration.

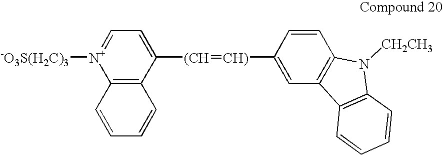

### Example 5

### Synthesis of Compounds 11-14

General scheme: a 4-amino-tetrafluorobenzaldehyde is refluxed with an equivalent of the corresponding 1-(3-bromopropyl)-4-methylpyridinium bromide in the presence of catalytic amount of piperidine overnight to obtain the target bromo intermediate which is subsequently displaced by triethylamine to generate the product.

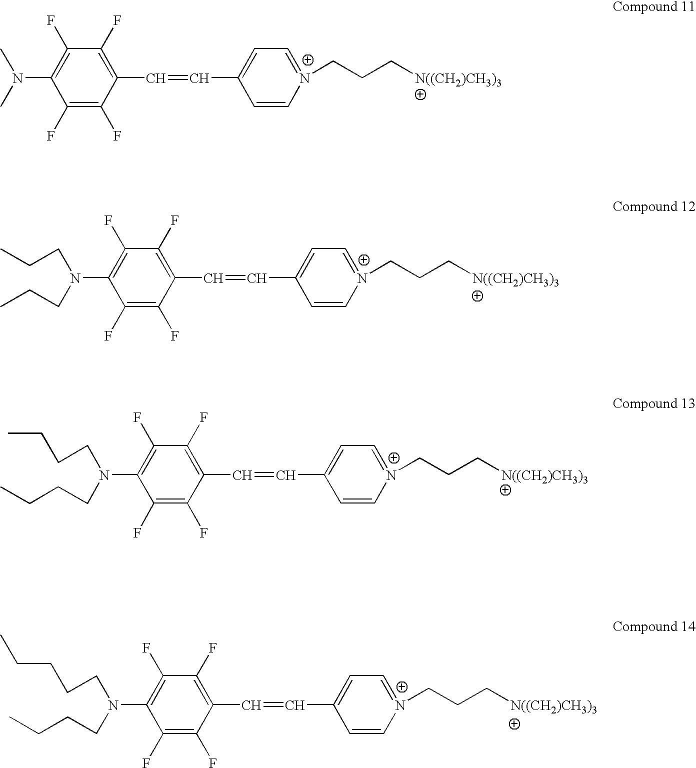

### Example 6

### Synthesis of Compounds 7, 8, 17, 18 and 21

General scheme: a 4-amino-tetrafluorobenzaldehyde is refluxed with an equivalent of the corresponding 2 or 4-methyl-1-(3-sulfobutyl)-pyridinium inner salt in the presence of catalytic amount of piperidine overnight to obtain the product.

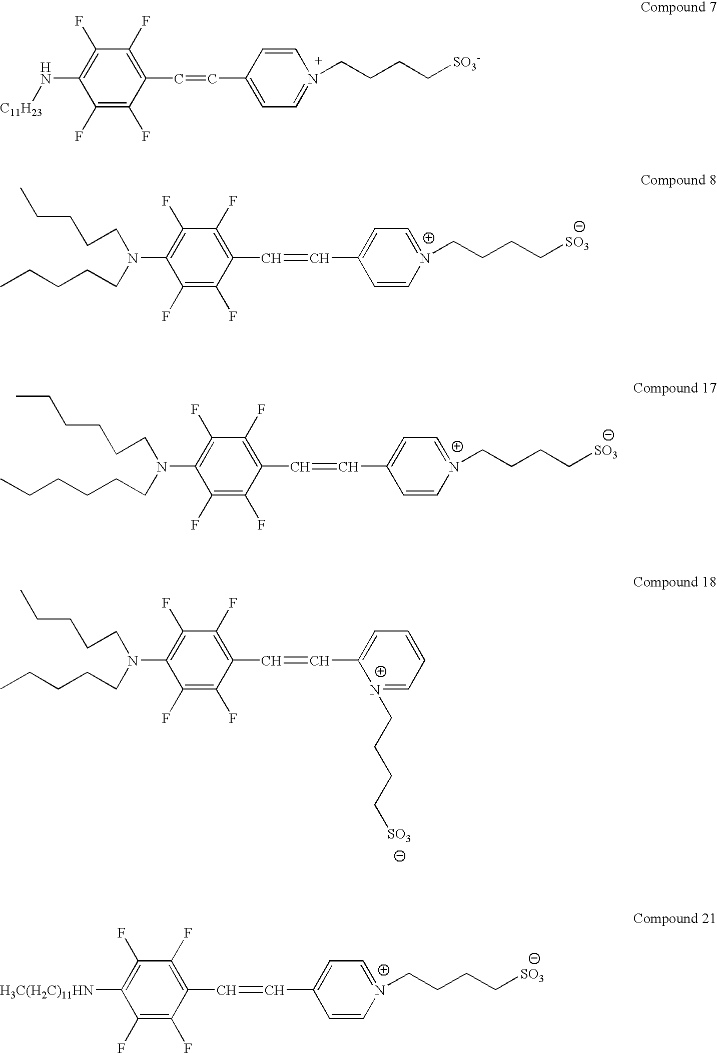

What is claimed is:

1) A staining solution for selectively detecting proteins that contain two or more alpha- helical transmembrane domains in a sample, wherein the staining solution comprises:
a) a lipophilic dye compound; and
b) at least about 30% (v/v) hydrophobic solvent.

2) The staining solution according to claim 1, wherein the lipophilic dye is a merocyanine dye, a cyanine dye, a styryl dye or a carbazolylvinyl dye.

3) The staining solution according to claim 1, wherein the dye compound has the general formula: A-B-E
wherein A is a nitrogen heterocycle;
B is a bridge moiety; and
E is an electron pair accepting moiety that comprises either a carbonyl or nitrogen atom.

4) The staining solution according to claim 3, wherein the dye is a cyanine dye.

5) The staining solution according to claim 3, wherein the dye is a merocyanine dye.

6) The staining solution according to claim 3, wherein the dye is a carbazolylvinyl dye.

7) The staining solution according to claim 3, wherein A is 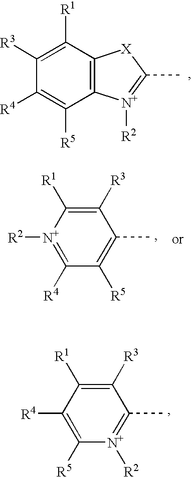
wherein,
R¹ is hydrogen, halogen, substituted halogen, alkyl, substituted alkyl, sulfoalkyl, alkoxy, substituted alkoxy, amino, substituted amino, aminoalkyl or substituted aminoalkyl;
R² is alkyl, substituted alkyl, sulfoalkyl, aminoalkyl, substituted aminoalkyl, sulfoalkyl or substituted sulfoalkyl;
R³ is hydrogen, halogen, substituted halogen, alkyl, substituted alkyl, sulfoalkyl, alkoxy, substituted alkoxy, amino, substituted amino, aminoalkyl or substituted aminoalkyl;
R⁴ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
R⁵ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
or a member independently selected from;
R¹ in combination with R³;
R³ in combination with R⁴; and
R⁴ in combination with R⁵;
together with the atoms to which they are joined, form a ring which is a 5-, 6- or 7-membered cycloalkyl, a 5-, 6- or 7-membered heterocycloalkyl, a 5-, 6- or 7-membered aryl or a 5-, 6- or 7-membered heteroaryl; and
X is O, S, NR⁵, or CR¹¹R¹² wherein R¹¹ is hydrogen, halogen, phenyl, substituted phenyl, substituted halogen, alkyl, or substituted alkyl; R¹² is hydrogen, halogen, phenyl, substituted phenyl, substituted halogen, alkyl, substituted alkyl; or R¹¹ and R¹² in combination form a 5- or 6-membered ring;
B is a covalent bridge having the formula —(CR¹¹═CR¹²)ₙ—;
wherein R¹¹ is hydrogen, halogen, phenyl, substituted phenyl, substituted halogen, alkyl, or substituted alkyl;
R¹² is hydrogen, halogen, phenyl, substituted phenyl, substituted halogen, alkyl, or substituted alkyl;
or R¹¹ and R¹² in combination form a 5- or 6-membered ring;
n is 1, 2 or 3;
E is 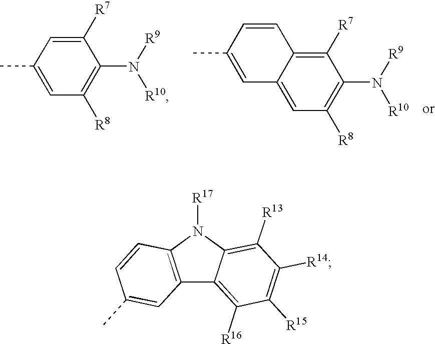
wherein R⁷ is hydrogen, halogen, substituted halogen, alkyl, substituted alkyl, sulfoalkyl, amino, substituted amino, aminoalkyl or substituted aminoalkyl;
R⁸ is hydrogen, halogen, substituted halogen, alkyl, substituted alkyl, sulfoalkyl, amino, substituted amino, aminoalkyl or substituted aminoalkyl;
R⁹ is alkyl, substituted alkyl, sulfoalkyl, aminoalkyl or substituted aminoalkyl;
R¹⁰ is alkyl, substituted alkyl, sulfoalkyl, aminoalkyl or substituted aminoalkyl; or
R⁹ and R¹⁰ in combination form a 5- or 6-membered ring, R⁹ and R⁷ in combination for a 5- or 6-membered ring or R¹⁰ and R⁸ in combination form a 5- or 6-membered ring;
R¹³ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁴ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁵ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁶ is hydrogen, halogen, substituted halogen, alkyl, substituted alkyl, sulfoalkyl, alkoxy, substituted alkoxy, amino, substituted amino, aminoalkyl or substituted aminoalkyl; and
R¹⁷ is alkyl, substituted alkyl, phenyl, substituted phenyl, amino alkyl, substituted aminoalkyl, sulfoalkyl, or substituted sulfoalkyl.

8. The staining solution according to claim 7, wherein the dye has the general formula:

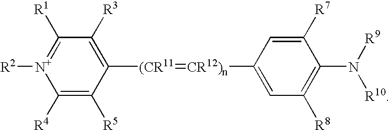

9. The staining solution according to claim 7, wherein the dye is:

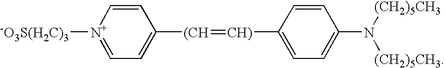

10. The staining solution according to claim 7, wherein the dye has the general formula:

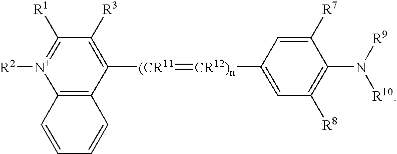

11. The staining solution according to claim 7, wherein the dye is:

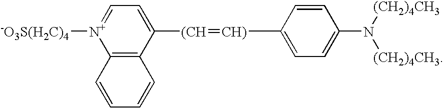

12. The staining solution according to claim 7, wherein the dye has the general formula:

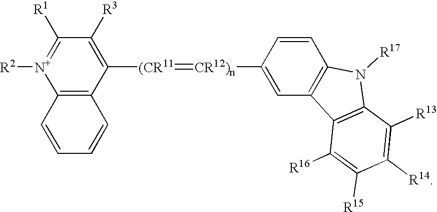

13. The staining solution according to claim 7, wherein the dye is

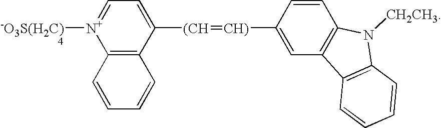

14. The staining solution according to claim 1, wherein the hydrophobic solvent is an alcohol, acetone, acetonitrile, or N-methyl pyrrolidone.

15. The staining solution according to claim 14, wherein the alcohol is methanol, isopropanol or ethanol.

16. The staining solution according to claim 1, wherein the staining solution comprises about 40% to about 75% hydrophobic solvent and about 0.5 μM to about 5 μM dye.

17. The staining solution according to claim 1, further comprising about 5% to about 30% acid.

18. The staining solution according to claim 17, wherein the acid is acetic acid.

19. The staining solution according to claim 17, wherein the acid is present in the staining solution at about 12% to about 20%.

20. The staining solution according to claim 1, further comprising HEPES at about pH 6.8.

21. The staining solution according to claim 16, wherein in the hydrophobic solvent is alcohol.

22. The staining solution according to claim 21, wherein the alcohol is ethanol, isopropanol or methanol.

23. The staining solution according to claim 21, wherein the alcohol is present in the staining solution at about 50%.

24. The staining solution according to claim 16, wherein the staining solution contains about 50% methanol, about 15% acetic acid and about 2 μM dye.

25. The staining solution according to claim 24, wherein the dye is

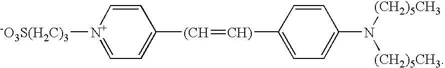

26. A staining solution for selectively detecting proteins that contain two or more alpha-helical transmembrane domains in a sample, wherein the staining solution comprises:
a) a lipophilic dye compound having the formula: 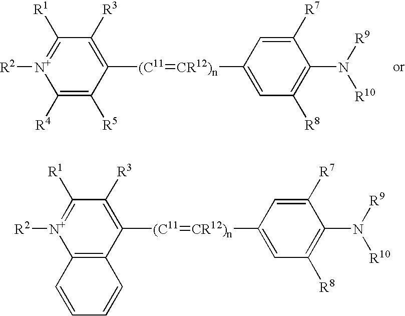
wherein
R¹ is hydrogen, halogen, substituted halogen, alkyl, substituted alkyl, sulfoalkyl, alkoxy, substituted alkoxy, amino, substituted amino, aminoalkyl or substituted aminoalkyl;
R² is alkyl, substituted alkyl, sulfoalkyl, substituted sulfoalkyl, aminoalkyl or substituted aminoalkyl;
R³ is hydrogen, halogen, substituted halogen, alkyl, substituted alkyl, sulfoalkyl, alkoxy, substituted alkoxy, amino, substituted amino, aminoalkyl or substituted aminoalkyl;
R⁴ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
R⁵ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
or a member independently selected from;
R¹ in combination with R³;
R³ in combination with R⁴; and
R⁴ in combination with R⁵;
together with the atoms to which they are joined, form a ring which is a 5-, 6- or 7-membered cycloalkyl, a 5-, 6- or 7-membered heterocycloalkyl, a 5-, 6- or 7-membered aryl or a 5-, 6- or 7-membered heteroaryl; and
n is 1, 2, or 3;
R¹¹ is hydrogen, halogen, phenyl, substituted phenyl, substituted halogen, alkyl, or substituted alkyl;
R¹² is hydrogen, halogen, phenyl, substituted phenyl, substituted halogen, alkyl, or substituted alkyl;
or R¹¹ and R¹² in combination form a 5- or 6-membered ring;
R⁷ is hydrogen, halogen, substituted halogen, alkyl, substituted alkyl, sulfoalkyl, amino, substituted amino, aminoalkyl or substituted aminoalkyl;
R⁸ is hydrogen, halogen, substituted halogen, alkyl, substituted alkyl, sulfoalkyl, amino, substituted amino, aminoalkyl or substituted aminoalkyl;
R⁹ is alkyl, substituted alkyl, sulfoalkyl, aminoalkyl or substituted aminoalkyl;
R¹⁰ is alkyl, substituted alkyl, sulfoalkyl, aminoalkyl or substituted aminoalkyl; or
R⁹ and R¹⁰ in combination form a 5- or 6-membered ring, R⁹ and R⁷ in combination for a 5- or 6-membered ring or R¹⁰ and R⁸ in combination form a 5- or 6-membered ring; and
b) at least about 30% (v/v) hydrophobic solvent.

27. The staining solution according to claim 26, further comprising an acid or HEPES.

28. The staining solution according to claim 26, wherein the staining solution comprises about 40% to about 75% hydrophobic solvent and about 0.5 μM to about 5 μM dye.

29. The staining solution according to claim 28, wherein the hydrophobic solvent is an alcohol, acetone, acetonitrile, or N-methyl pyrrolidone.

30. The staining solution according to claim 29, wherein the alcohol is methanol, isopropanol or ethanol.

31. The staining solution according to claim 28, wherein the staining solution contains about 50% methanol, about 15% acetic acid and about 2 μM dye.

32. The staining solution according to claim 31, wherein the dye is:

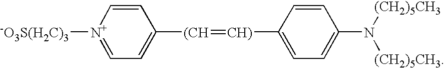

33. A method for selective detection of proteins that comprise two or more alpha-helical transmembrane domains, wherein the method comprises:
a) contacting a sample with a staining solution for selectively detecting proteins that comprise two or more alpha-helical transmembrane domains to prepare a labeled sample mixture, wherein the staining solution comprises
i. a lipophilic dye compound; and
ii. at least about 30% (v/v) hydrophobic solvent;
b) incubating the labeled sample mixture for a sufficient amount of time to allow the lipophilic dye compound of the staining solution to bind to a protein in the sample that comprises two or more alpha-helical transmembraneous domains;
c) illuminating the incubated sample mixture with an appropriate wavelength to form an illuminated sample; and
d) observing the illuminated sample, wherein a protein that comprises two or more alpha-helical transmembrane domains is detected in the illuminated sample.

34. The method according to claim 33, wherein the sample is immobilized on a polymeric membrane, within a polyacrylamide gel, within an agarose gel, on a polymeric membrane or on a microarray, before the incubated sample mixture is illuminated.

35. The method according to claim 33, wherein the sample is electrophoretically separated in a polymeric medium before the incubated sample mixture is illuminated.

36. The method according to claim 35, wherein the polymeric medium comprises SDS-and polyacrylamide.

37. The method according to claim 33, wherein the sample is separated by capillary electrophoresis before the incubated sample mixture is illuminated.

38. The method according to claim 33, wherein the sample further comprises a detergent before the sample is contacted with the staining solution.

39. The method according to claim 38, wherein the detergent is an anionic detergent.

40. The method according to claim 39, wherein the anionic detergent is an alkyl sulfate having about 6-18 carbons, or an alkyl sulfonate salt having about 6-18 carbons.

41. The method according to claim 40, wherein the anionic detergent is sodium dodecyl sulfate (SDS).

42. The method according to claim 33, further comprising preparing an aqueous cell-free sample mixture, wherein any biological membranes present in the sample have been removed, destroyed, or dispersed below a critical micelle concentration before the sample is contacted with the staining solution.

43. A method for selective detection of proteins that contain two or more alpha-helical transmembrane domains in a sample, wherein the method comprises:
a) electrophoretically separating the sample on an SDS-polyacrylamide gel to prepare an immobilized sample;
b) removing the SDS from the polyacrylamide gel to prepare an essentially SDS-free polyacrylamide gel;
c) contacting the essentially SDS-free polyacrylamide gel with a staining solution for selectively detecting proteins that contain two or more alpha-helical transmembrane domains in a sample to prepare a stained sample, wherein the staining solution comprises
i. a lipophilic dye compound; and
ii. at least about 30% (v/v) hydrophobic solvent;
d) illuminating the stained sample with an appropriate wavelength; and
e) observing the illuminated sample whereby the protein that contains two or more alpha-helical transmembrane domains is detected.

44. The method according to claim 43, wherein removing the SDS comprises contacting the sample with a fixing solution, wherein the fixing solution comprises an alcohol and an acid.

45. The method according to claim 43, further comprising washing the stained sample to remove unbound dye before the sample has been illuminated.

46. The method according to claim 43, further comprising preparing an aqueous cell-free sample mixture, wherein any biological membranes present in the sample have been removed, destroyed, or dispersed below a critical micelle concentration before the sample is contacted with the staining solution.

47. A kit for selective detection of proteins that contain two or more alpha-helical transmembrane domains in a sample, wherein the kit comprises: a staining solution for selectively detecting proteins that contain two or more alpha-helical transmembrane domains in a sample, wherein the staining solution comprises
i. a lipophilic dye compound; and
ii. at least about 30% (v/v) hydrophobic solvent;

48. The kit according to claim 47, further comprising instructions for selective detection of proteins that contain two or more alpha-helical transmembrane domains in a sample.
